(19) United States
(12) Patent Application Publication
(10) Pub. No.: US 2022/0233159 A1
(43) Pub. Date: Jul. 28, 2022

(54) MEDICAL IMAGE PROCESSING METHOD AND DEVICE USING MACHINE LEARNING

(71) Applicant: INDUSTRIAL COOPERATION FOUNDATION CHONBUK NATIONAL UNIVERSITY, Jeollabuk-do (KR)

(72) Inventors: Sun Jung YOON, Jeollabuk-do (KR); Min Woo KIM, Jeollabuk-do (KR); II Seok OH, Jeollabuk-do (KR); Kap Soo HAN, Jeollabuk-do (KR); Myoung Hwan KO, Jeollabuk-do (KR); Woong CHOI, Gyeonggi-do (KR)

(21) Appl. No.: 17/614,890

(22) PCT Filed: Feb. 28, 2020
(86) PCT No.: PCT/KR2020/002866

§ 371 Date: Nov. 29, 2021

(30) Foreign Application Priority Data
May 29, 2019 (KR) 10-2019-0063078

(51) Int. Cl. A61B 6/00; G16H 30/40; G16H 50/20; G06T 7/00; G06V 10/25; G06V 10/774

(52) U.S. Cl. CPC A61B 6/5217; A61B 6/505; A61B 6/469; G16H 30/40; G16H 50/20; G06T 2207/30008; G06V 10/25; G06V 10/774; G06T 2207/10116; G06T 2207/20081; G06T 7/0014

(57) ABSTRACT

A medical image processing method using machine learning according to an embodiment of the present invention includes acquiring an X-ray image of an object, identifying a plurality of anatomical regions by applying a deep learning technique for each bone structure region that constitutes the X-ray image, predicting a bone disease according to bone quality for each of the plurality of anatomical regions, and determining an artificial joint that replaces the anatomical region in which the bone disease is predicted.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2020/002866, filed Feb. 28, 2020, which claims priority to the benefit of Korean Patent Application No. 10-2019-0063078 filed in the Korean Intellectual Property Office on May 29, 2019, the entire contents of which are incorporated herein by reference.

### BACKGROUND

### 1. Technical Field

The present disclosure relates to a medical image processing method and device using machine learning in which human musculoskeletal tissues in a medical image are identified by machine learning and distinguishably displayed in color to determine the size of an artificial joint (implant) that replaces the musculoskeletal tissue more accurately.

In addition, the present disclosure relates to a medical image processing method and device using machine learning in which the diameter and roundness of the femoral head are numerically inferred by comparing the femoral head identified by predicting femoroacetabular impingement syndrome (FAI) from an X-ray image with a pre-registered femoral head from the deep learning technique in a repeated manner.

### 2. Background Art

When performing a lower limb hip joint surgery, to increase the accuracy of the surgery, a surgeon analyzes the shape of tissues (bones and joints) in acquired x-ray images, and preoperatively plans (templating) the size and type of an artificial joint (implant) to be applied in the surgery.

For example, in the case of the hip joint, the surgeon identifies the size and shape of the socket of the joint part and the bone part (femoral head, stem, etc.) in the x-ray images, indirectly measures using the template of the artificial joint to apply, selects the artificial joint that fits the size and shape and uses it in the surgery.

As described above, only an indirect method that determines the size and shape of the artificial joint to be used in the surgery in reliance on the surgeon's subject determination has been adopted, and there may be a difference between the size/shape of the prepared artificial joint and the actually necessary size/shape in the actual surgery, resulting in low accuracy of the surgery and the prolonged operative time.

To solve the problem, some foreign artificial joint companies provide their own programs to support artificial joint surgeries, but do not publish or open to the public, and the technical levels of the programs are so low that there are many restrictions for surgeons to use.

Accordingly, there is an urgent need for a new technology for anatomically identify the type of tissue according to image brightness by analysis of medical images, to allow surgeons to correctly know the positions and shapes of patients' joints.

### SUMMARY

An embodiment of the present disclosure is directed to providing a medical image processing method and device using machine learning, in which anatomical regions in a patient's image are identified considering the bone structure, and a bone disease is predicted for each identified anatomical region, thereby facilitating the determination of an artificial joint to be used in surgery.

In addition, an embodiment of the present disclosure is aimed at matching color to each identified anatomical region and displaying to allow a surgeon to easily visually perceive the individual anatomical regions.

In addition, an embodiment of the present disclosure is aimed at presenting the sphericity of the femoral head through prediction and outputting to an X-ray image even though parts of the femoral head are abnormally shaped due to femoroacetabular impingement syndrome (FAI), thereby providing medical support for the reconstruction of the damaged hip joint close to the shape of the normal hip joint in fracture surgery and arthroscopy.

A medical image processing method using machine learning according to an embodiment of the present disclosure includes acquiring an X-ray image of an object, identifying a plurality of anatomical regions by applying a deep learning technique for each bone structure region that constitutes the X-ray image, predicting a bone disease according to bone quality for each of the plurality of anatomical regions, and determining an artificial joint that replaces the anatomical region in which the bone disease is predicted.

In addition, a medical image processing device using machine learning according to an embodiment of the present disclosure includes an interface unit to acquire an X-ray image of an object, a processor to identify a plurality of anatomical regions by applying a deep learning technique for each bone structure region that constitutes the X-ray image, and predict a bone disease according to bone quality for each of the plurality of anatomical regions, and a computation controller to determine an artificial joint that replaces the anatomical region in which the bone disease is predicted.

According to an embodiment of the present disclosure, it is possible to provide a medical image processing method and device using machine learning, in which anatomical regions in a patient's image are identified considering the bone structure, and a bone disease is predicted for each identified anatomical region, thereby facilitating the determination of an artificial joint to be used in surgery.

In addition, according to an embodiment of the present disclosure, color is matched to each identified anatomical region and displayed to allow a surgeon to easily visually perceive the individual anatomical regions.

### BRIEF DESCRIPTION OF THE DRAWINGS

FIG. 1 is a block diagram showing the internal configuration of a medical image processing device using machine learning according to an embodiment of the present disclosure.

FIG. 2 is a diagram showing an example of anatomical regions according to deep learning segmentation.

FIG. 3 is a diagram illustrating an example of a result of segmentation by the application of a trained deep learning technique.

FIGS. 4A and 4B are diagrams illustrating a manual template that has been commonly used in hip joint surgery.

FIGS. 5A and 5B are diagrams showing an example of a result of auto templating by the application of a trained deep learning technique according to the present disclosure.

FIG. 6 is a flowchart illustrating a process of predicting an optimal size and shape of an artificial joint according to the present disclosure.

FIGS. 7A and 7B are diagrams illustrating an example of presenting the sphericity of the femoral head having femoroacetabular impingement syndrome (FAI) through an X-ray image and calibrating an aspherical region using Burr according to the present disclosure.

FIG. 8 is a flowchart showing the flow of a medical image processing method according to an embodiment of the present disclosure.

### DETAILED DESCRIPTION

Hereinafter, embodiments will be described in detail with reference to the accompanying drawings. However, a variety of modification may be made to the embodiments and the scope of protection of the patent application is not limited or restricted by the embodiments. It should be understood that all modifications, equivalents or substitutes to the embodiments are included in the scope of protection.

The terminology used in an embodiment is for the purpose of describing the present disclosure and is not intended to be limiting of the present disclosure. Unless the context clearly indicates otherwise, the singular forms include the plural forms as well. The term “comprises” or “includes” when used in this specification, specifies the presence of stated features, integers, steps, operations, elements, components or groups thereof, but does not preclude the presence or addition of one or more other features, integers, steps, operations, elements, components or groups thereof.

Unless otherwise defined, all terms (including technical and scientific terms) used herein have the same meaning as commonly understood by those having ordinary skill in the technical field to which the embodiments belong. It will be understood that terms, such as those defined in commonly used dictionaries, should be interpreted as having a meaning that is consistent with their meaning in the context of the relevant art document, and will not be interpreted in an idealized or overly formal sense unless expressly so defined herein.

Additionally, in describing the present disclosure with reference to the accompanying drawings, like reference signs denote like elements irrespective of the drawing symbols, and redundant descriptions are omitted. In describing the embodiments, when a detailed description of relevant known technology is determined to unnecessarily obscure the subject matter of the embodiments, the detailed description is omitted.

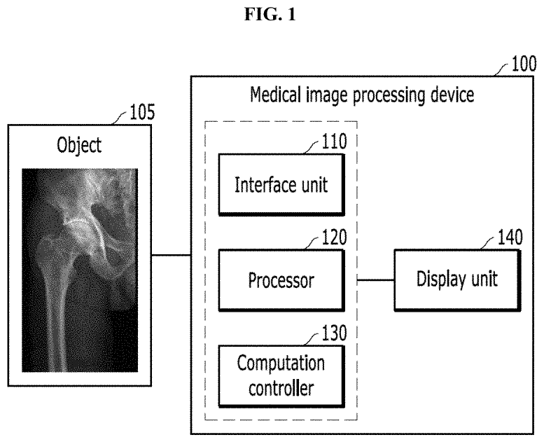

FIG. 1 is a block diagram showing the internal configuration of a medical image processing device using machine learning according to an embodiment of the present disclosure.

Referring to FIG. 1, the medical image processing device 100 according to an embodiment of the present disclosure may include an interface unit 110, a processor 120 and a computation controller 130. Additionally, according to embodiments, the medical image processing device 100 may further include a display unit 140.

To begin with, the interface unit 110 acquires an X-ray image of an object 105. That is, the interface unit 110 may be a device that irradiates X-ray for diagnosis onto the object 105 or a patient, and acquires a resulting image as the X-ray image. The X-ray image is an image showing the bone structure that blocks the passage of the X-ray beam through the human body, and may be commonly used to diagnose the bone condition of the human body through a to clinician's clinical determination. The diagnosis of the bone by the X-ray image may be, for example, joint dislocation, ligament injuries, bone tumors, calcific tendinitis determination, arthritis, bone diseases, etc.

The processor 120 identifies a plurality of anatomical regions by applying the deep learning technique for each bone structure region that constitutes the X-ray image. Here, the bone structure region may refer to a region in the image including a specific bone alone, and the anatomical region may refer to a region determined to need surgery in a bone structure region.

That is, the processor 120 may play a role in identifying the plurality of bone structure regions uniquely including the specific bone by analysis of the X-ray image, and identifying the anatomical region as a surgery range for each of the identified bone structure regions.

The deep learning technique may refer to a technique for mechanical data processing by extracting useful information by analysis of previous accumulated data similar to data to be processed. The deep learning technique shows the outstanding performance in image recognition, and is evolving to assist clinicians in diagnosis in the applications of image analysis and experimental result analysis in the health and medical field.

The deep learning in the present disclosure may assist in extracting an anatomical region of interest from the bone structure region based on the previous accumulated data.

That is, the processor 120 may define a region occupied by the bone in the X-ray image as the anatomical region by interpreting the X-ray image by the deep learning technique.

In the anatomical region identification, the processor 120 may identify the plurality of anatomical regions by distinguishing the bone quality according to the radiation dose of the bone tissue with respect to the bone structure region. That is, the processor 120 may detect the radiation dose of each bone of the object 105 by image analysis, predict the composition of the bone according to the detected radiation dose, and identify the anatomical region in which the surgery is to be performed.

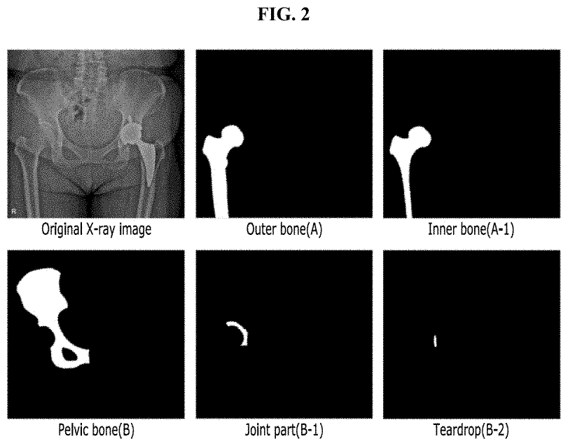

For example, FIG. 2 described below shows identifying a bone structure region including at least a left leg joint part from an original image, and identifying five anatomical structures (femur A, inner femur A-1, pelvic bone B, joint part B-1, teardrop B-2), considering the radiation dose of an individual bone tissue, with respect to the identified bone structure region.

Additionally, the processor 120 may predict a bone disease according to the bone quality for each of the plurality of anatomical regions. That is, the processor 120 may predict the bone condition from the anatomical region identified as a region of interest and diagnose a disease that the corresponding bone is suspected of having. For example, the processor 120 may predict fracture in the joint part by detecting a difference/unevenness exhibiting a sharp change in brightness in the joint part, i,e., the anatomical region,

Additionally, the computation controller 130 may determine an artificial joint that replaces the anatomical region in which the bone disease is predicted. The computation controller 130 may play a role in determining the size and shape of the artificial joint to be used in the surgery when the bone disease is predicted for each anatomical region.

In determining the artificial joint, the computation controller 130 may determine the shape and size of the artificial joint based on the shape and size (ratio) of the bone disease.

To this end, the computation controller 130 may detect the shape and ratio occupied by the bone disease in the anatomical region in which the bone disease is predicted. That is, the computation controller 130 may recognize the outer shape of the bone disease presumed to have occurred in the bone and the size of the bone disease occupied in the bone and represent as an image. In an embodiment, when the occupation ratio of the bone disease is high (when the bone disease occurs in most of the bone), the computation controller 130 may detect the entire anatomical region in which the bone disease is predicted.

Additionally, the computation controller 130 may search for a candidate artificial joint having a contour that matches the detected shape within a preset range in a database. That is, the computation controller 130 may search, as the candidate artificial joint, an artificial joint that matches the shape of the bone occupied by the bone disease among a plurality of artificial joints kept in the database after training,

Subsequently, the computation controller 130 may determine the shape and size of the artificial joint by selecting, as the artificial joint, a candidate artificial joint within a predetermined range from the size calculated by applying a specified weight to the detected ratio from the found candidate artificial joints. That is, the computation controller 130 may calculate the actual size of the bone disease by multiplying the size of the bone disease in the X-ray image by the weight set according to the image resolution, and select a candidate artificial joint similar to the calculated actual size of the bone disease.

For example, when the image resolution of the X-ray image is 50%, the computation controller 130 may calculate the actual size of ‘10 cm’ of the bone disease by applying multiplication to the size of ‘5 cm’ of the bone disease in the X-ray image by the weight of ‘2’ according to the image resolution of 50%, and determine the candidate artificial joint that generally matches the actual size of ‘10 cm’ of the bone disease as the artificial joint that replaces the anatomical region in which the bone disease is predicted.

According to an embodiment, the medical image processing device 100 of the present disclosure may further include the display unit 140 to output the X-ray image processed according to the present disclosure.

To begin with, the display unit 140 may numerically represent the cortical bone thickness according to parts of the bone belonging to the bone structure region, and output to the X-ray image. That is, the display unit 140 may play a role in measuring the cortical bone thickness of a specific region within the bone in the X-ray image, including the measured value in the X-ray image and outputting it. In an embodiment, the display unit 140 may visualize by tagging the measured cortical bone thickness with the corresponding bone part in the X-ray image.

Additionally, the display unit 140 may extract name information corresponding to the contour of each of the plurality of anatomical regions from a training table. That is, the display unit 140 may extract the name information defining the identified anatomical region of interest according to similarity of shape.

Subsequently, the display unit 140 may associate the name information to each anatomical region and output to the X-ray image. That is, the display unit 140 may play a role in including the extracted name information in the X-ray image and outputting it. In an embodiment, the display unit 140 may visualize by tagging the extracted name information with the corresponding bone part in the X-ray image, to allow not only the surgeon but also ordinary people to easily know the name of each bone included in the X-ray image.

Additionally, the display unit 140 may identify the plurality of anatomical regions by matching color to each anatomical region and outputting to the X-ray image, and in this instance, may match at least different colors to adjacent anatomical regions. That is, the display unit 140 may visually identify the identified anatomical regions by overlaying with different colors in a sequential order, to allow the surgeon to perceive each anatomical region more intuitively.

According to an embodiment of the present disclosure, it is possible to provide a medical image processing method and device using machine learning, in which anatomical regions in a patient's image are identified considering the bone structure, and a bone disease is predicted for each identified anatomical region, thereby facilitating the determination of an artificial joint to be used in surgery.

Additionally, according to an embodiment of the present disclosure, color is matched to each identified anatomical region and displayed to allow a surgeon to easily visually perceive the individual anatomical regions.

FIG. 2 is a diagram showing an example of the anatomical regions according to deep learning segmentation.

The medical image processing device 100 of the present disclosure anatomically identifies the type of tissue according to image brightness by analysis of an X-ray image and performs pseudo-coloring.

Additionally, the medical image processing device 100 improves the accuracy of anatomical tissue identification based on the pseudo-coloring technique by applying the machine learning technique. Additionally, the medical image processing device 100 may set the size of an artificial joint (cup and stem) to be applied based on the shape and size of the identified tissue. Through this, the medical image processing device 100 assists in reconstructing a surgery site closest to an anatomically normal health part.

As shown in FIG. 2, the medical image processing device 100 may segment an original X-ray image into five anatomical regions by applying the deep learning technique. That is, the medical image processing device 100 may segment the anatomical regions of outer bone A, inner bone A-1, pelvic bone B, joint part B-1 and Teardrop B-2 from the original X-ray image.

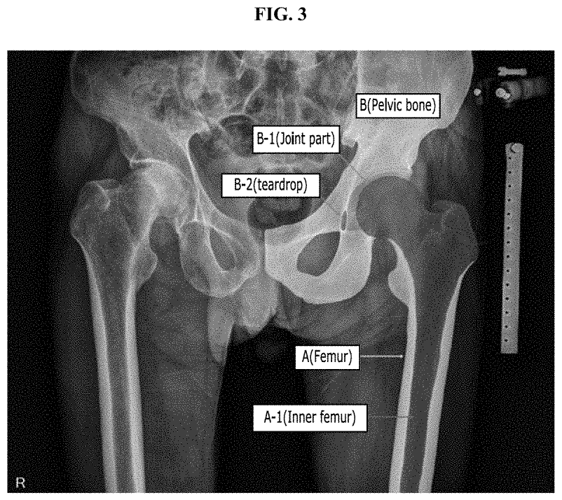

FIG. 3 is a diagram illustrating an example of a result of segmentation by the application of the trained deep learning technique.

FIG. 3 shows an output X-ray image in which color is matched to each anatomical region identified from the X-ray image. That is, the medical image processing device 100 matches pelvic bone B-yellow,joint part B-1-orange, Teardrop B-2-pink, outer bone (femur) A-green and inner bone (inner femur) A-1-blue on the X-ray image, and outputs it.

In this instance, the medical image processing device 100 may match at least different colors to adjacent anatomical regions. In FIG. 3, for example, the medical image processing device 100 may match different colors, yellow and orange, to the pelvic bone B and the joint part B-1 adjacent to each other, to allow the surgeon to intuitively identify the anatomical regions.

Additionally, the medical image processing device 100 may associate name information to each anatomical region and output as the X-ray image. FIG. 3 shows connecting the name information of the pelvic bone B to the anatomical region corresponding to the pelvic bone and displaying on the X-ray image.

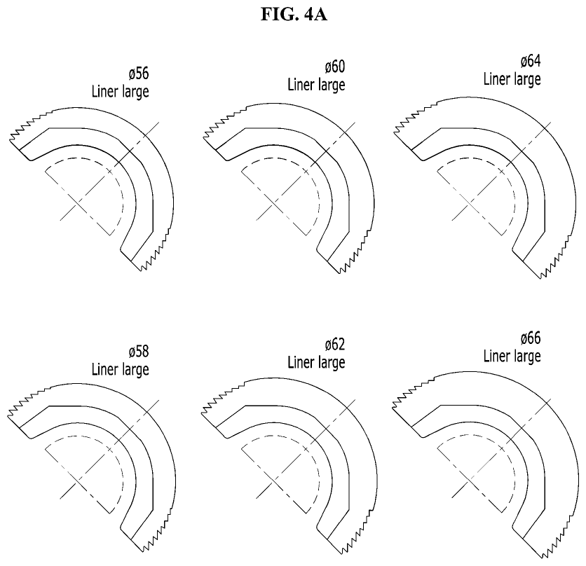

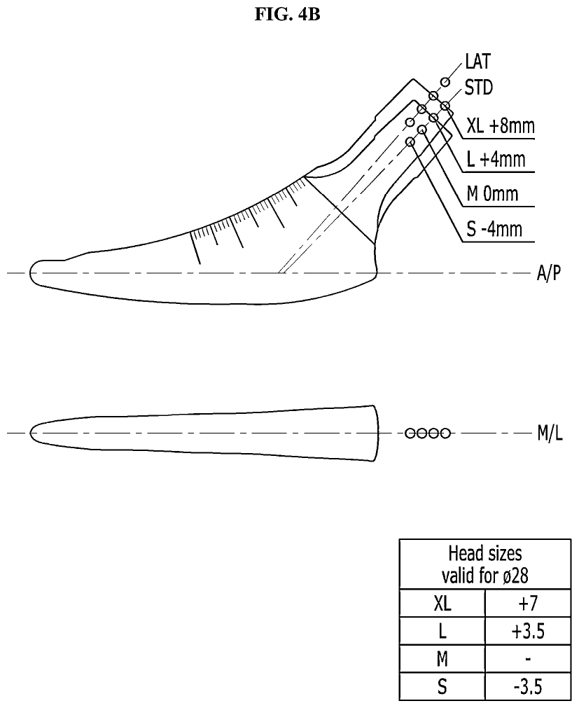

FIGS. 4A and 4B are diagrams showing a manual template that has been commonly used in hip joint surgery.

FIG. 4A shows a cup template for an artificial hip joint, and FIG. 4B shows an artificial joint stem template. The template may be a preset standard scaler to estimate the size and shape of an anatomical region to be replaced.

Through the template, a surgeon may determine the size and shape of an artificial joint that will replace the anatomical region in which the bone disease is suspected.

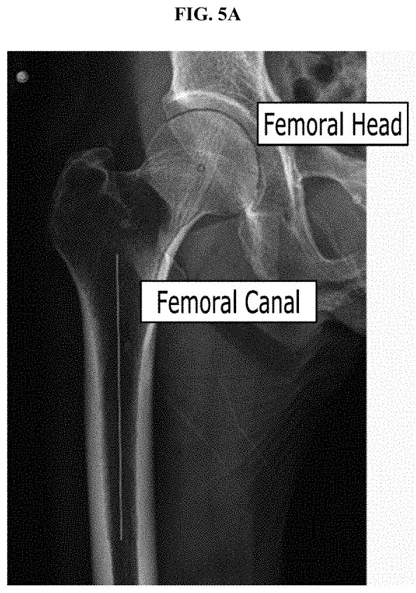

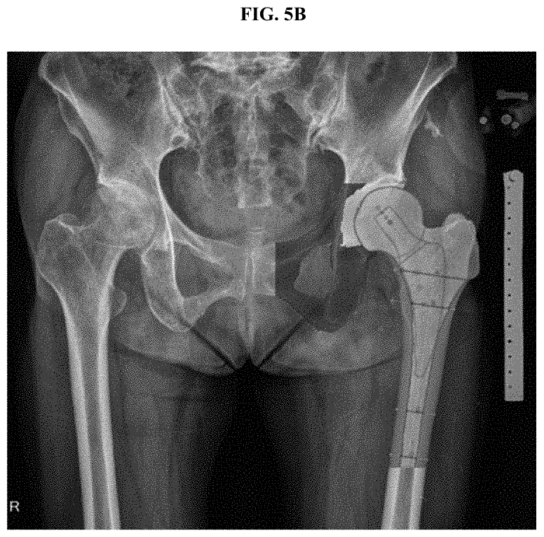

FIGS. 5A and 5B are diagrams showing an example of a result of auto templating by the application of the trained deep learning technique according to the present disclosure.

As shown in FIGS. 5A and 5B, the medical image processing device 100 of the present disclosure may automatically determine the artificial joint that replaces the anatomical region in which the bone disease is predicted. FIG. 5A shows the femoral canal and the femoral head identified as the anatomical region, and FIG. 5B shows an image of the artificial joint that matches the shape and size of the femoral canal and the femoral head, automatically determined through the processing in the present disclosure and displayed on the X-ray image.

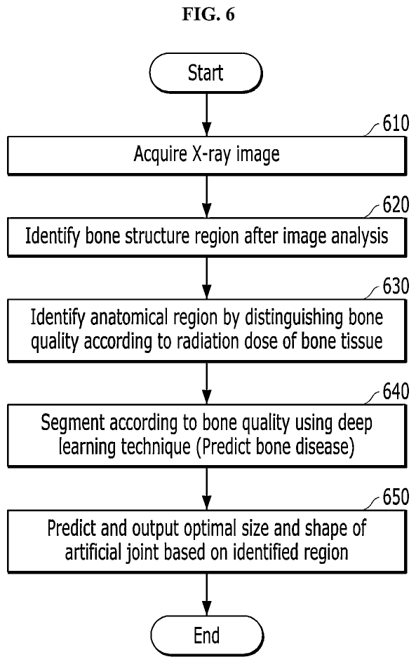

FIG. 6 is a flowchart illustrating a process of predicting an optimal size and shape of the artificial joint according to the present disclosure.

To begin with, the medical image processing device 100 may acquire the X-ray image (610). That is, the medical image processing device 100 may acquire the X-ray image by capturing the bone structure of the object 105.

Additionally, the medical image processing device 100 may identify the bone structure region after image analysis (620). That is, the medical image processing device 100 may separate the bone structure region that constitutes the X-ray image. In this instance, the medical image processing device 100 may develop the deep learning technique for measuring the size of the bone structure.

Additionally, the medical image processing device 100 may identify the anatomical region by distinguishing the bone quality according to the radiation dose of the bone tissue (630). That is, the medical image processing device 100 may identify the anatomical region by distinguishing the bone quality (normal/abnormal) according to the radiation dose of the bone tissue using the developed technique. For example, as shown in FIGS. 2 and 3 described previously, the medical image processing device 100 may segment into the anatomical regions of outer bone A, inner bone A-1, pelvic bone B, joint part B-1, and Teardrop B-2.

Subsequently, the medical image processing device 100 may segment according to the bone quality using the deep learning technique (640). That is, the medical image processing device 100 may predict the bone disease according to the bone quality after image analysis by using the deep learning technique.

Additionally, the medical image processing device 100 may predict and output the optimal size and shape of the artificial joint based on the identified region (650). That is, the medical image processing device 100 may automatically match the artificial joint to the region in which the bone disease is predicted, and output the optimal size and shape of the matched artificial joint, As an example of auto templating, the medical image processing device 100 may automatically determine an image of the artificial joint that matches the shape and size the femoral canal and the femoral head, and display on the X-ray image, as shown in FIGS. 4A, 4B, 5A and 5B described previously.

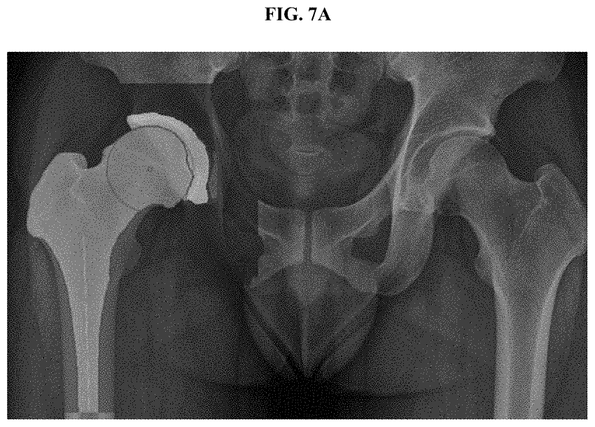

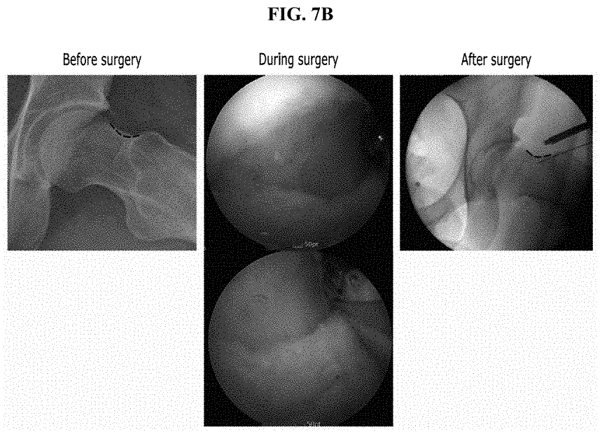

Hereinafter, an example of the present disclosure of reconstructing into the shape of the normal hip joint by calculating the sphericity of the femoral head will be described through FIGS. 7A and 7B.

FIGS. 7A and 7B are diagrams showing an example of presenting the sphericity of the femoral head having femoroacetabular impingement syndrome (FAI) through the X-ray image and calibrating an aspherical region using Burr according to the present disclosure.

FIG. 7A shows an image displaying sphericity for the anatomicalregion in which the bone disease is predicted.

As a result of predicting the bone disease according to the bone quality, when the anatomical region in which the bone disease is predicted is femoral head, the processor 120 may estimate the diameter and roundness of the femoral head by applying the deep learning technique.

Here, the femoral head is a region corresponding to the top of the femur which is the thighbone, and may refer to a round part located at the upper end of the femur.

Additionally, the diameter of the femoral head may refer to an average length from the center of the round part to the edge,

Additionally, the roundness of the femoral head may refer to a numerical representation of how much the round part is close to a circle,

That is, the processor 120 may numerically infer the diameter and roundness of the femoral head by comparing the femoral head identified by predicting femoroacetabular impingement syndrome (FAI) from the X-ray image with the pre-registered femoral head from the deep learning technique in a repeated manner.

Additionally, the processor 120 predicts a circular shape for the femoral head based on the estimated diameter and roundness. That is, the processor 120 may predict the current shape of the femoral head damaged by femoroacetabular impingement syndrome (FAI) through the previously estimated diameter/roundness.

FIG. 7A shows that a part of the femoral head has an imperfect circular shape due to femoroacetabular impingement syndrome (FAI) induced by the damage of the femoral head indicated in green. Additionally, FIG. 7A shows the perfect shape of the femoral head having no bone disease as the circular dotted line.

Subsequently, the display unit 140 may display the region of the femoral head including asphericity from the predicted circular shape by an indicator, and output to the X-ray image. That is, the display unit 140 may display the arrow as the indicator in the region having no perfect circular shape due to the damage, and map on the X-ray image and output it.

The region of the femoral head indicated by the arrow in FIG. 7A may refer to the starting point of asphericity, i.e., a point of loss of sphericity of the femoral head.

When a clinician receives the X-ray image of FIG. 7A, the clinician visually perceives the damaged part of the femoral head to be reconstructed during arthroscopy while directly seeing the current shape of the femoral head with an eye.

FIG. 7B shows images of the femoral head before and after calibration according to the present disclosure in arthroscopy for femoroacetabular impingement syndrome (FAI).

FIG. 7B illustrates an example of comparing and displaying the shape of the femoral head before and after surgery in the calibration of the aspherical abnormal region of the femoral head close to the spherical shape using Burr in arthroscopy of FAI.

Through this, by the present disclosure, it is possible to provide not only artificial joint templating but also medical support for the reconstruction of the damaged hip joint close to the shape of the normal hip joint in fracture surgery and arthroscopy.

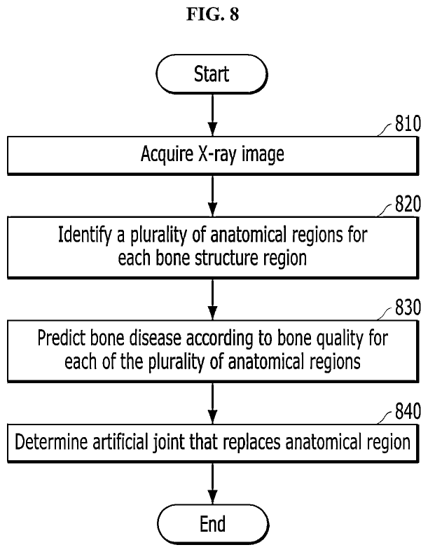

Hereinafter, FIG. 8 details the work flow of the medical image processing device 100 according to embodiments of the present disclosure.

FIG, 8 is a flowchart showing the flow of a medical image processing method according to an embodiment of the present disclosure.

The medical image processing method according to this embodiment may be performed by the above-described medical image processing device 100 using machine learning,

To begin with, the medical image processing device 100 acquires an X-ray image of an object (810). This step 810 may be a process of irradiating X-ray for diagnosis onto the object or a patient, and acquiring a resulting image as the X-ray image, The X-ray image is an image showing the bone structure that blocks the passage of the X-ray beam through the human body, and may be commonly used to diagnose the bone condition of the human body through a clinician's clinical determination. The diagnosis of the bone by the X-ray image may be, for example, joint dislocation, ligament injuries, bone tumors, calcific tendinitis determination, arthritis, bone diseases, etc.

Additionally, the medical image processing device 100 identifies a plurality of anatomical regions by applying the deep learning technique for each bone structure region that constitutes the X-ray image (820). Here, the bone structure region may refer to a region in the image including a specific bone alone, and the anatomical region may refer to a region determined to need surgery in a bone structure region.

The step 820 may be a process of identifying the plurality of bone structure regions uniquely including the specific bone by analysis of the X-ray image, and identifying the anatomical region as a surgery range for each of the identified bone structure regions.

The deep learning technique may refer to a technique for mechanical data processing by extracting useful information by analysis of previous accumulated data similar to data to be processed, The deep learning technique shows the outstanding performance in image recognition, and is evolving to assist clinicians in diagnosis in the applications of image analysis and experimental result analysis in the health and medical field.

The deep learning in the present disclosure may assist in extracting an anatomical region of interest from the bone structure region based on the previous accumulated data.

That is, the medical image processing device 100 may define a region occupied by the bone in the X-ray image as the anatomical region by interpreting the X-ray image by the deep learning technique.

In the anatomical region identification, the medical image processing device 100 may identify the plurality of anatomical regions by distinguishing the bone quality according to the radiation dose of the bone tissue with respect to the bone structure region. That is, the medical image processing device 100 may detect the radiation dose of each bone of the object by image analysis, predict the composition of the bone according to the detected radiation dose, and identify the anatomical region in which the surgery is to be performed.

For example, the medical image processing device 100 may identify a bone structure region including at least a left leg joint part from an original image, and identify five anatomical structures (femur A, inner femur A-1, pelvic bone B, joint part B-1, teardrop B-2), considering the radiation dose of the individual bone tissue, with respect to the identified bone structure region.

Additionally, the medical image processing device 100 may predict a bone disease according to the bone quality for each of the plurality of anatomical regions (830). The step 830 may be a process of predicting the bone condition from the anatomical region identified as a region of interest and diagnosing a disease that the corresponding bone is suspected of having, For example, the medical image processing device 100 may predict fracture in the joint part by detecting a difference/unevenness exhibiting a sharp change in brightness in the joint part, i.e., the anatomical region.

Additionally, the medical image processing device 100 determines an artificial joint that replaces the anatomical region in which the bone disease is predicted (840). The step 840 may be a process of determining the size and shape of the artificial joint to be used in the surgery for each anatomical region when the bone disease is predicted.

In determining the artificial joint, the medical image processing device 100 may determine the shape and size of the artificial joint based on the shape and size (ratio) of the bone disease.

To this end, the medical image processing device 100 may detect the shape and ratio occupied by the bone disease in the anatomical region in which the bone disease is predicted. That is, the medical image processing device 100 may recognize the outer shape of the bone disease presumed to have occurred in the bone and the size of the bone disease occupied in the bone, and represent as an image. In an embodiment, when the occupation ratio of the bone disease is high (when the bone disease occurs in most of the bone), the medical image processing device 100 may detect the entire anatomical region in which the bone disease is predicted.

Additionally, the medical image processing device 100 may search for a candidate artificial joint having a contour that matches the detected shape within a preset range in the database. That is, the medical image processing device 100 may search, as the candidate artificial joint, an artificial joint that matches the shape of the bone occupied by the bone disease among a plurality of artificial joints kept in the database after training.

Subsequently, the medical image processing device 100 may determine the shape and size of the artificial joint by selecting, as the artificial joint, a candidate artificial joint within a predetermined range from the size calculated by applying a specified weight to the detected ratio among the found candidate artificial joints, That is, the medical image processing device 100 may calculate the actual size of the bone disease by multiplying the size of the bone disease in the X-ray image by the weight set according to the image resolution, and select the candidate artificial joint close to the calculated actual size of the bone disease.

For example, when the image resolution of the X-ray image is 50%, the medical image processing device 100 may calculate the actual size of ‘10 cm’ of the bone disease by applying multiplication to the size of ‘5 cm’ of the bone disease in the X-ray image by the weight of ‘2’ for the image resolution of 50%, and determine the candidate artificial joint that generally matches the actual size of ‘10 cm’ of the bone disease as the artificial joint that replaces the anatomical region in which the bone disease is predicted.

Additionally, the medical image processing device 100 may numerically represent the cortical bone thickness according to parts of the bone belonging to the bone structure region, and output to the X-ray image. That is, the medical image processing device 100 may measure the cortical bone thickness of a specific region within the bone in the X-ray image, include the measured value in the X-ray image and output it. In an embodiment, the medical image processing device 100 may visualize by tagging the measured cortical bone thickness with the corresponding bone part in the X-ray image.

Additionally, the medical image processing device 100 may extract name information corresponding to the contour of each of the plurality of anatomical regions from the training table. That is, the medical image processing device 100 may extract the name information defining the identified anatomical region of interest according to similarity of shape.

Subsequently, the medical image processing device 100 may associate the name information to each anatomical region and output to the X-ray image. That is, the medical image processing device 100 may play a role in including the extracted name information in the X-ray image and outputting it. In an embodiment, the medical image processing device 100 may visualize by tagging the extracted name information with the corresponding bone part in the X-ray image, to allow not only the surgeon but also ordinary people to easily know the name of each bone included in the X-ray image.

Additionally, the medical image processing device 100 may identify the plurality of anatomical regions by matching color to each anatomical region and outputting to the X-ray image, and in this instance, may match at least different colors to adjacent anatomical regions. That is, the medical image processing device 100 may visually identify the identified anatomical regions by overlaying with different colors in a sequential order, to allow the surgeon to perceive each anatomical region more intuitively.

The method according to an embodiment may be implemented in the format of program instructions that may be executed through a variety of computer means and recorded in computer readable media. The computer readable media may include program instructions, data files and data structures alone or in combination. The program instructions recorded in the media may be specially designed and configured for embodiments or known and available to persons having ordinary skill in the field of computer software. Examples of the computer readable recording media include hardware devices specially designed to store and execute the program instructions, for example, magnetic media such as hard disk, floppy disk and magnetic tape, optical media such as CD-ROM and DVD, magneto-optical media such as floptical disk, and ROM, RAM and flash memory. Examples of the program instructions include machine code generated by a compiler as well as high-level language code that can be executed by a computer using an interpreter. The hardware device may be configured to act as one or more software modules to perform the operation of embodiments, and vice versa.

The software may include computer programs, code, instructions, or a combination of at least one of them, and may enable a processing device to work as desired or command the processing device independently or collectively. The software and/or data may be permanently or temporarily embodied in a certain type of machine, component, physical equipment, virtual equipment, computer storage medium or device or transmitted signal wave to be interpreted by the processing device or provide instructions or data to the processing device. The software may be distributed on computer systems connected via a network, and stored or executed in a distributed manner. The software and data may be stored in at least one computer readable recording medium.

Although the embodiments have been hereinabove described by a limited number of drawings, it is obvious to those having ordinary skill in the corresponding technical field that a variety of technical modifications and changes may be applied based on the above description. For example, even if the above-described technologies are performed in different sequences from the above-described method, and/or the components of the above-described system, structure, device and circuit may be connected or combined in different ways from the above-described method or may be replaced or substituted by other components or equivalents, appropriate results may be attained.

Therefore, other implementations, other embodiments and equivalents to the appended claims fall within the scope of the appended claims.

1. A medical image processing method using machine learning, comprising:
acquiring an X-ray image of an object;
identifying a plurality of anatomical regions by applying a deep learning technique for each bone structure region that constitutes the X-ray image;
predicting a bone disease according to bone quality for each of the plurality of anatomical regions; and
determining an artificial joint that replaces the anatomical region in which the bone disease is predicted.

2. The medical image processing method using machine learning according to claim 1, wherein the identifying of the plurality of the anatomical regions comprises identifying the plurality of anatomical regions by distinguishing the bone quality according to a radiation dose of a bone tissue with respect to the bone structure region.

3. The medical image processing method using machine learning according to claim 1, wherein the determining of the artificial joint comprises:
detecting a shape and ratio occupied by the bone disease in the anatomical region in which the bone disease is predicted;
searching for a candidate artificial joint having a contour that matches the detected shape within a preset range in a database; and
determining a shape and size of the artificial joint by selecting, as the artificial joint, a candidate artificial joint within a predetermined range from a size calculated by applying a specified weight to the detected ratio among the found candidate artificial joints.

4. The medical image processing method using machine learning according to claim 1, further comprising:
numerically representing a cortical bone thickness according to parts of a bone belonging to the bone structure region, and outputting to the X-ray image.

5. The medical image processing method using machine learning according to claim 1, further comprising:
extracting name information corresponding to a contour of each of the plurality of anatomical regions from a training table; and
associating the name information to each anatomical region and outputting to the X-ray image.

6. The medical image processing method using machine learning according to claim 1, further comprising:
matching color to each anatomical region and outputting to the X-ray image to identify the plurality of anatomical regions, wherein at least different colors are matched to adjacent anatomical regions.

7. The medical image processing method using machine learning according to claim 1, further comprising:
when the anatomical region in which the bone disease is predicted is a femoral head, estimating a diameter and roundness of the femoral head by applying the deep learning technique;
predicting a circular shape for the femoral head based on the estimated diameter and roundness; and
displaying a region of the femoral head including asphericity from the predicted circular shape by an indicator, and outputting to the X-ray image.

8. A medical image processing device using machine learning, comprising:
an interface unit to acquire an X-ray image of an object;
a processor to identify a plurality of anatomical regions by applying a deep learning technique for each bone structure region that constitutes the X-ray image, and predict a bone disease according to bone quality for each of the plurality of anatomical regions; and
a computation controller to determine an artificial joint that replaces the anatomical region in which the bone disease is predicted.

9. The medical image processing device using machine learning according to claim 8, wherein the processor identifies the plurality of anatomical regions by distinguishing the bone quality according to a radiation dose of a bone tissue with respect to the bone structure region.

10. The medical image processing device using machine learning according to claim 8, wherein the computation controller is configured to detect a shape and ratio occupied by the bone disease in the anatomical region in which the bone disease is predicted, search for a candidate artificial joint having a contour that matches the detected shape within a preset range in a database, and determine a shape and size of the artificial joint by selecting, as the artificial joint, a candidate artificial joint within a predetermined range from a size calculated by applying a specified weight to the detected ratio among the found candidate artificial joints.

11. The medical image processing device using machine learning according to claim 8, further comprising:
a display unit to numerically represent a cortical bone thickness according to parts of a bone belonging to the bone structure region, and output to the X-ray image.

12. The medical image processing device using machine learning according to claim 8, further comprising:
a display unit to extract name information corresponding to a contour of each of the plurality of anatomical regions from a training table, associate the name information to each anatomical region and output to the X-ray image.

13. The medical image processing device using machine learning according to claim 8, further comprising:
a display unit to match color to each anatomical region and output to the X-ray image to identify the plurality of anatomical regions, wherein at east different colors are matched to adjacent anatomical regions.

14. The medical image processing device using machine learning according to claim 8, wherein when the anatomical region in which the bone disease is predicted is a femoral head, the processor estimates a diameter and roundness of the femoral head by applying the deep learning technique, predicts a circular shape for the femoral head based on the estimated diameter and roundness, displays a region of the femoral head including asphericity from the predicted circular shape by an indicator through a display unit, and outputs to the X-ray image.
